(19) United States
(12) Patent Application Publication
(10) Pub. No.: US 2015/0105671 A1
(43) Pub. Date: Apr. 16, 2015

(54) BIOLOGICAL INFORMATION MEASUREMENT DEVICE

(71) Applicant: Seiko Epson Corporation, Shinjuku-ku (JP)

(72) Inventors: Junya SHIBUYA, Azumino-shi (JP); Yoshihiro Hidai, Shiojiri-shi (JP); Shigemitsu Tanaka, Okaya-shi (JP)

(21) Appl. No.: 14/511,138

(22) Filed: Oct. 9, 2014

(30) Foreign Application Priority Data
Oct. 11, 2013 (JP) 2013-213478

(51) Int. Cl. A61B 5/024

(52) U.S. Cl. CPC A61B 5/02427
USPC 600/479

(57) ABSTRACT

A biological information measurement device includes a device main body and a band configured to secure the device to a body part of a user. The device main body contacts the user's skin to thereby acquire biological information about the user, and presents a non-rectangular parallelogram surface in plan view. The parallelogram surface has two longitudinal edges disposed along a longitudinal direction, and two oblique edges disposed along an oblique direction that is transverse to, but not perpendicular to, the longitudinal direction. The band is attached to the oblique edges of the device main body, and generally extends in the longitudinal direction.

### CROSS REFERENCE TO RELATED APPLICATIONS

The present application is based on and claims priority from Japanese Patent Application No. 2013-213478, filed Oct. 11, 2013, the disclosure of which is incorporated herein by reference.

### BACKGROUND

1. Technical Field

The present invention relates to a biological information measurement device which is worn by a user so as to measure biological information, such as the user's heart rate.

2. Related Art

A wrist-worn heart rate monitor generally includes a device main body, which contains the necessary electronics, and a wrist band which secures the monitor to the user's wrist. It is necessary to bring the device main body into close contact with the user's skin, such as with a tightly worn elastic band which secures the monitor tightly around the wrist. A band with a stretchable elastic belt portion is disclosed in JP-A-2012-90975, which is incorporated herein by reference.

The band described in JP-A-2012-90975 is provided with first and second band members which are mounted on a device main body, and a connecting member. Each band member has a stretchable portion which expands and contracts along a longitudinal direction.

When mounting such a biological information measurement device on the wrist of a test object, a tensile force acts on the band by an operation of a wearer, and a device main body is mounted on the wrist in a state where the stretchable portion is extended by the tensile force. Then, if the wearer removes the band from the hand, a restoring force acts on the stretchable portion. In this way, the band is tightened up, and thus it is possible to bring the device main body into close contact with the wrist regardless of the degree of tightening of the device main body to the wrist by the wearer.

However, the band is thinner than the width of the device main body, and therefore, the user perceives the band as creating excessive pressure to the point of feeling discomfort. The user may therefore remove the device sooner than he or she otherwise would, which detracts from the amount of useful information that can be gleaned. Further, the device main body is large and therefore heavy. Therefore, an inertial force acting when a test object moves is higher than it otherwise would be, further adding to the user's discomfort. Still further, a large display surface helps the visual recognition of the measured biological information, but detracts from battery life.

### SUMMARY

A biological information measurement device includes a device main body and a band configured to secure the device to a body part of a user. The device main body contacts the user's skin to thereby acquire biological information about the user, and presents a non-rectangular parallelogram surface in plan view. The parallelogram surface has two longitudinal edges disposed along a longitudinal direction, and two oblique edges disposed along an oblique direction that is transverse to, but not perpendicular to, the longitudinal direction. The band is attached to the oblique edges of the device main body, and generally extends in the longitudinal direction.

The device may further include a mounting member which connects the device main body and the band.

The device may further include a bottomed groove between the band and one of the oblique edges of the device main body.

The device main body may further include a case and a module located within the case. The case may include a front surface to be viewed by the user and a rear surface to contact the user's skin. The module may include an electronic circuit. A film including metal may be provided on the front surface of the case, where the film and the module are electrically connected.

The module may include a first display portion presenting a rectangular surface in plan view. The rectangular surface have have two longitudinal edges which are collinear with respective ones of the longitudinal edges of the parallelogram surface, and two lateral edges disposed perpendicular to the longitudinal direction.

Each of the lateral edges of the rectangular surface may define an acute angle at an intersection with a respective one of the oblique edges of the parallelogram surface.

The module may further include a second display portion adjacent the first display portion.

The second display portion may present a right triangular surface in plan view.

The module may include a control section, and an operation section connected to the control section. The operation section may be manipulable by the user and disposed on one of the longitudinal edges.

The module may include a power supply section with a battery which drives at least one of the control section, the first display portion, and the second display portion. The device may further include at least one output terminal to output data from the control section to outside the device, connected to the module and located on one of the longitudinal edges. The device may further include at least one charging terminal to charge the battery, connected to the module and located on one of the longitudinal edges.

There may be a first output terminal, a second output terminal, a first charging terminal, and a second charging terminal, where the distance between the first and the second charging terminals is different than the distance between the first and the second output terminals.

The terminals may be located along the longitudinal edge in a row in the following order: the first charging terminal, the first output terminal, the second output terminal, the second charging terminal.

Each of the terminals may include stainless steel.

Tips of the terminals may be exposed on a surface of the case.

The case may further include a positioning hole disposed in the same row as the terminals.

A method of measuring biological information of a user includes measuring the biological information using a device that closely contacts the user's skin; and presenting a non-rectangular parallelogram surface of the device to the user to thereby alleviate a sense of discomfort caused by the perception of excessive pressure.

### BRIEF DESCRIPTION OF THE DRAWINGS

The invention will be described with reference to the accompanying drawings, wherein like numbers reference like elements.

FIG. 1 is a top view schematically showing the external appearance of a biological information measurement device according to an embodiment.

FIGS. 2A and 2B are perspective views schematically showing the external appearance of the biological information measurement device according to the embodiment.

FIG. 3 is a development view schematically showing the structure of the biological information measurement device according to the embodiment.

FIG. 4 is an enlarged view schematically showing the structure of a main body section of the biological information measurement device.

FIGS. 5A and 5B are side views schematically showing the nine o′clock-side side surface of the main body section of the biological information measurement device.

FIGS. 6A and 6B are a plan view and a cross-sectional view schematically showing a first band portion.

FIGS. 7A to 7C are a plan view and a cross-sectional view schematically showing a second band portion.

FIGS. 8A and 8B are schematic views illustrating the perceived size of the display area.

### DESCRIPTION OF EXEMPLARY EMBODIMENTS

Hereinafter, an exemplary embodiment of the invention will be described using the drawings, which are not necessarily to scale. To the extent that relative sizes and/or shapes of components are considered relevant, these will be explicitly mentioned in the description.

### Schematic Configuration of Biological Information Measurement Device 1

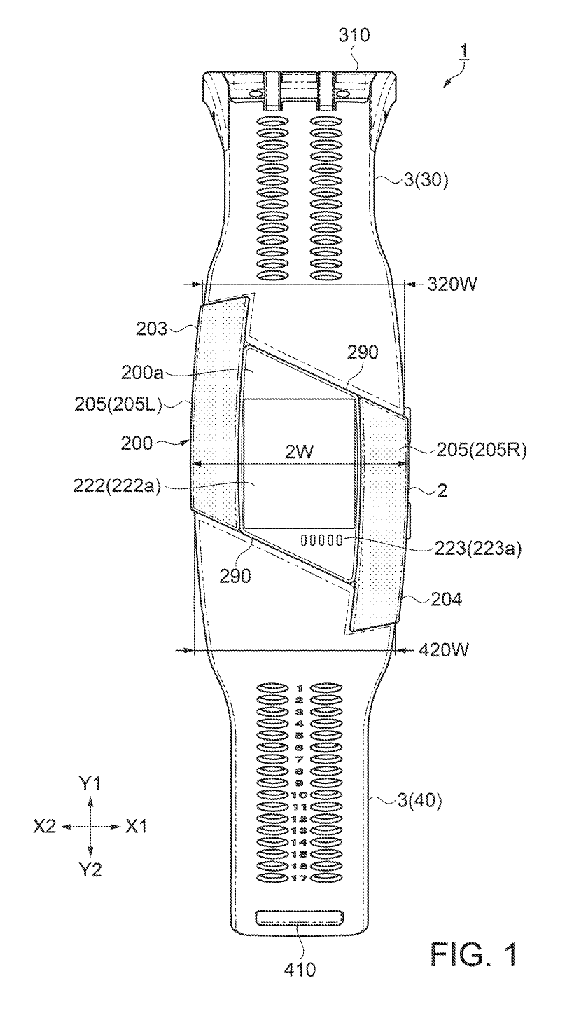

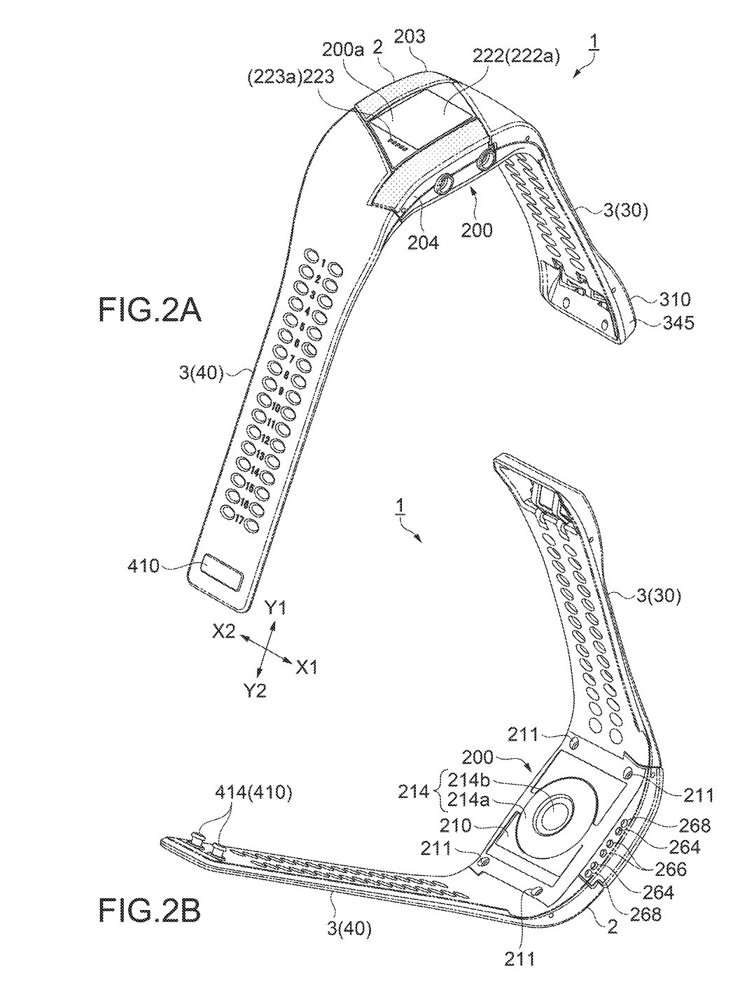

A biological information measurement device 1 is an electronic device which can be worn on the wrist or other body part of a user to measure biological information of the user, such as the pulse rate. The measurement device 1 generally resembles a wristwatch, as shown in FIGS. 1, 2A, and 2B. The measurement device 1 includes a device main body 2 which includes the electronics and measures the biological information by contacting the user's skin, and a band 3 which is mounted on the device main body 2 and serves to attach the device 1 to the user's wrist or other body part.

### Configuration of Device Main Body 2

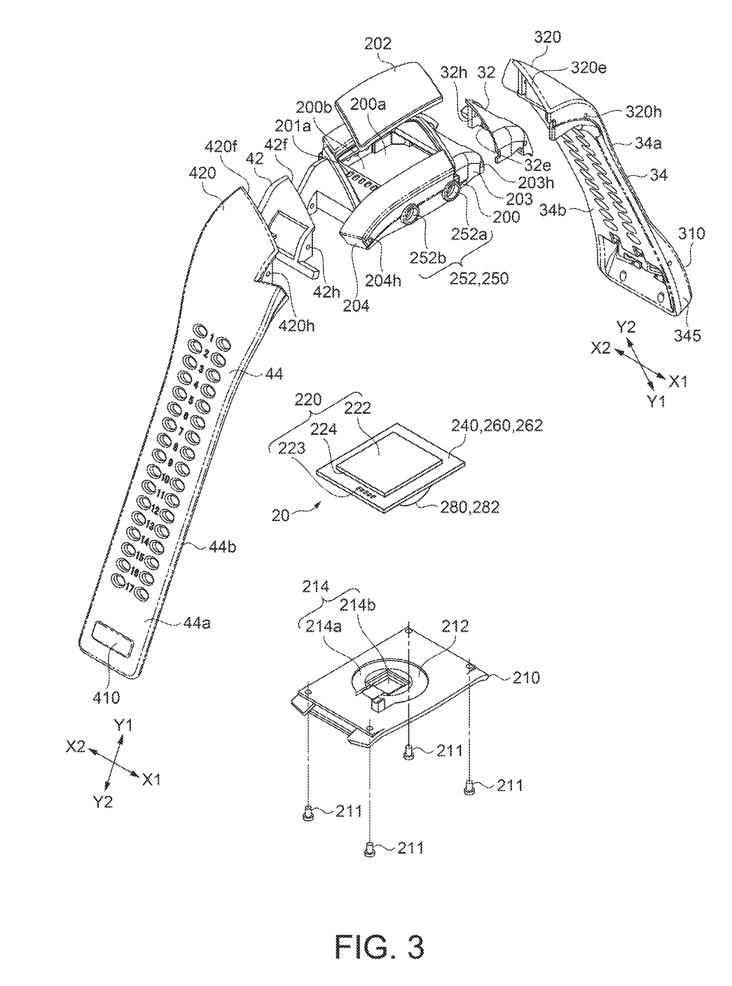

As shown in FIGS. 2A, 2B, and 3, the device main body 2 of the measurement device 1 is provided with a module 20 (FIG. 3), and a case 200 in which the module 20 is accommodated. The module 20 is provided with a display section 220, a processing section 240, a power supply section 260, and a sensor section 280.

In addition, the display section 220 is provided with a first display portion 222 and a second display portion 223. Further, the device main body 2 is provided with an operation section 250 connected to the processing section 240, and a communication terminal 266. In addition, the device main body 2 is provided with a charging terminal 264 connected to the power supply section 260.

In the case 200, a concave portion 200b in which the module 20 is accommodated is formed on the lid section 210 side thereof. The module 20 is accommodated in the concave portion 200b and the lid section 210 covers the concave portion 200b and is fixed by setscrews 211. Materials of the case 200 and the lid section 210 are not particularly limited. As an example thereof, nylon-based synthetic resin (plastic resin) may be used.

As shown in FIGS. 2A, 2B, and 3, in the case 200, a display window 200a is provided on the opposite side to the side on which the lid section 210 is provided. The display window 200a is configured such that the user's pulse rate or the like which is displayed on the display section 220 is visible through the window 200a.

The display window 200a is provided with a first display window 222a corresponding to a first display portion 222, and a second display window 223a corresponding to a second display portion 223. The first display portion 222 is fitted into the first display window 222a (refer to FIGS. 1, 2A, 2B, and 4). Further, the second display portion 223 is fitted into the second display window 223a (refer to FIGS. 1, 2A, 2B, and 4).

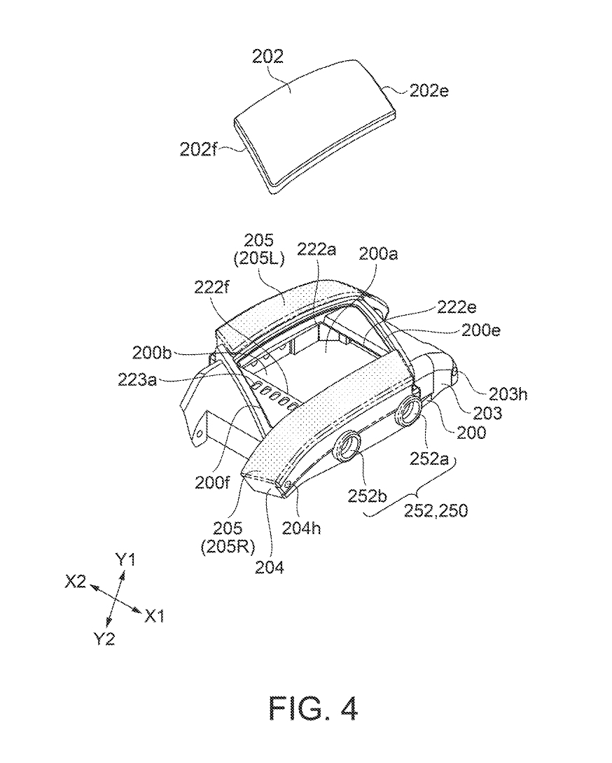

As shown in FIG. 4, the display window 200a has a non-rectangular parallelogram shape with two longitudinal edges disposed along the longitudinal direction of the device, and two oblique edges 200e, 200f disposed along an oblique direction that is transverse to, but not perpendicular to, the longitudinal direction.

The first display window 222a in which the first display portion 222 is fitted has a rectangular shape having sides 222e and 222f which extend in the lateral direction perpendicular to the longitudinal direction.

The second display window 223a in which the second display portion 223 is fitted has several parallel sub-windows, each with a corner-rounded rectangular shape aligned in the longitudinal direction.

The edge 200e of the display window 200a and the edge 222e of the first display window 222a define an acute angle therebetween. Further, the edge 200f of the display window 200a and the edge 222f of the first display window 222a define and acute angle therebetween.

The second display window 223a is disposed in the triangular region between the edge 200f of the display window 200a and the edge 222f of the first display window 222a.

The second display portions 223 display an operating state or the like of the measurement device 1 in a plurality of ways. Further, it is possible to make the first display portion 222 (the first display window 222a) appear larger than it actually is. Therefore, it is possible to suppress a feeling of pressure which is given to a person with the measurement device 1 mounted thereon.

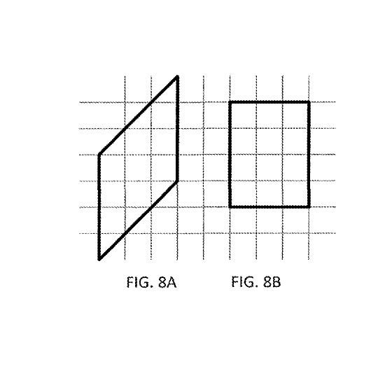

In other words, turning to FIGS. 8A and 8B, we see that the two shapes have identical areas: twelve squares each. However, the non-rectangular shape of FIG. 8A appears larger than the rectangle of FIG. 8B. Therefore, the non-rectangular parallelogram shape of the display window 200a gives the user the perception of it being larger than it actually is. The sense of a large area contacting the user's skin can lead the user to believe that the pressure is not excessive and that the device is not uncomfortable, without the need for actually making the display window 200a larger, which would lead to decreased battery life.

Further, a display section cover body 202 formed of transparent resin, transparent glass, or the like is fitted into the display window 200a. The display section 220 is protected by the display section cover body 202. The display section cover body 202 has a non-rectangular parallelogram shape, sized to be set in the display window 200a.

As shown in FIGS. 1 to 4, at the case 200, a frame section 205 is provided along the display window 200a.

In the frame section 205, a frame portion 205R is provided in an X1 direction (the three o′clock direction when comparing the device to a wristwatch), and a frame portion 205L is provided in an X2 direction (the nine o′clock direction).

Each frame portion 205R, 205L is shaped as an isosceles trapezoid with the bases disposed in the longitudinal direction, i.e. parallel to the edges of the band 3.

In the frame portion 205L, one of the legs of the trapezoid disposed in a Y2 direction is provided as an extension of the oblique line of the edge 200f of the display window 200a, and the other leg provided in a Y1 direction is provided further to the Y1 side (twelve o′clock) than an extension of the oblique line of the edge 200e of the display window 200a.

In the frame portion 205R, one of the legs of the trapezoid disposed in the Y1 direction is provided as an extension of the oblique line of the edge 200e of the display window 200a, and the other leg provided in the Y2 direction is provided further to the Y2 side (six o′clock) than an extension of the oblique line of the edge 200f of the display window 200a.

At the frame section 205, a film 205a which includes metal is provided on the front side of the case 200 in which the display window 200a is provided. The film 205a is electrically connected to the processing section 240 (described later). The film 205a can be used as an antenna when performing wireless communication with an information processing device such as the user's computer or mobile phone (not shown) provided outside the measurement device 1. Further, the film 205a can be a touch sensor and used as the operation section 250 (described later). In addition, the film 205a adds to the strength of the case 200 and allows the wall thickness of the case 200 to be thinner. Therefore, it is possible to keep the weight of the case 200 down.

The material of the film 205a is not particularly limited. In presently preferred embodiments, the material is capable of acting as an antenna and/or detecting a change in capacitance. In a particularly presently preferred embodiment, a material which includes nickel (Ni) is used. Nickel acts as an antenna, strengthens the case 200, acts as a touch switch, and has an attractive glossy color. In addition, a presently preferred embodiment, in order to increase performance as an antenna, the strength of the case 200, and the aesthetics, the film 205a is also provided on the side surface of the frame section 205.

As shown in FIG. 3, the display section 220 is part of the module 20 accommodated in the case 200. The first display portion 222 and the second display portion 223 are portions of the display section 220.

In the first display portion 222, biological information such as the number of pulses (i.e. heart beats), time information such as the current time, or the like is displayed depending on a selected display mode. Further, at the first display portion 222, a backlight 224 is provided and can illuminate the first display portion 222.

The first display portion 222 can include any suitable hardware capable of displaying biological information (such as a numeral or a graph configured by a dot matrix) such as the pulse rate. A liquid crystal display device is one example. Further, the backlight 224 can be any suitable light of any color. One example is an electro-luminescence (EL) panel which emits green light.

The second display portion 223 can be any suitable light of any color capable of indicating the operation mode or the like of the measurement device 1 with its color, on/off state, or blinking. One example is a light-emitting diode, or, as illustrated, several light-emitting diodes.

As shown in FIG. 3, the processing section 240 is provided in the module 20 accommodated in the case 200.

The processing section 240 includes a substrate configured as a semiconductor device such as a microcomputer or a storage device, and an electronic circuit or the like which communicates information. The display section 220, the operation section 250, the power supply section 260, the sensor section 280, and the film 205a of the frame section 205 are connected to the processing section 240. The processing section 240 processes the driving of the sensor section 280 or a signal received from the sensor section 280 (e.g. the pulse rate or another signal derived from the pulse rate) so that the pulse rate or other biological information can be displayed in the display section 220.

Further, the processing section 240 stores of biological information and communicates with the information processing device provided outside the measurement device 1, thereby outputting the stored data.

As shown in FIG. 3, the operation section 250 providing a command to the processing section 240 is provided in the module 20 accommodated in the case 200.

The operation section 250 includes a button 252 configured to be pressed by the user, Pressing the operation button 252 may. e.g. switch between modes, such as a pulse measurement mode of displaying pulse rate; a clock mode of displaying the current time, a stopwatch time, or the like; a remaining battery level display mode; a lighting mode of the backlight 224 of the display section 220; or the like.

The operation section 250 is provided in the side surface of the case 200 in the X1 direction (the three o′clock-side direction) shown in FIGS. 1 to 4. The operation section 250 can be provided with a plurality of operation buttons 252. It is presently preferred that at least one operation button 252 is provided on the extension line of the edge 200e of the display window 200a. In this way, the user visually recognizes the edge 200e of the display window 200a, whereby it is possible to easily grasp the position where the operation button 252 is provided, and thus it is possible to prevent an erroneous operation.

In a further presently preferred embodiment, pressing all the buttons 252 at substantially the same time switches to a setting mode to allow the user to set the measurement device 1. In the illustrated embodiment, two buttons 252a and 252b are provided. The button 252a can be pressed by an index finger and adding a force associated with the pressing to the case 200 through a thumb.

The button 252b can be pressed by a ring finger and adding a force associated with the pressing to the case 200 through a thumb. Therefore, it is preferable that an interval at which the operation buttons 252a and 252b are provided is an interval in which fingers (for example, a thumb and an index finger, or a thumb and a ring finger) performing the operation do not overlap, in a range that the fingers performing the operation reach.

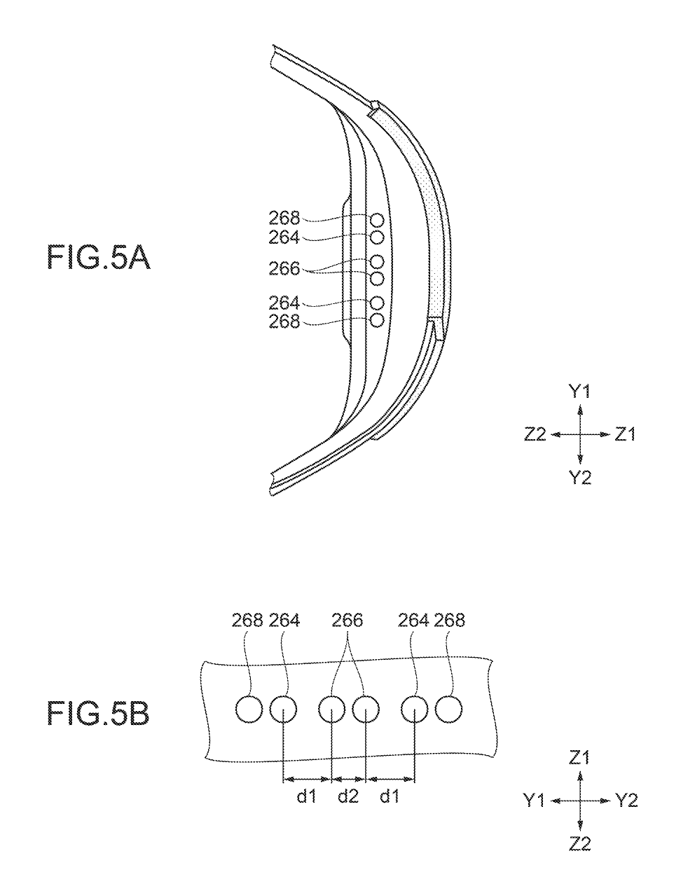

As shown in FIGS. 5A and 5B, the charging terminal 264 which is used in the charging of the battery 262 installed in the power supply section 260 of the measurement device 1 is provided in the case 200. In addition, the communication terminal 266 which is used to communicate biological information (data) measured and stored in the measurement device 1, measurement setting data, and the like is provided in the case 200.

The charging terminal 264 and the communication terminal 266 are provided in the side surface of the case 200 in the X2 direction (the nine o′clock direction). Each of the charging terminal 264 and the communication terminal 266 is provided as a single terminal or as a plurality of terminals, depending on design considerations. In the illustrated example, two charging terminals 264 and two communication terminals 266 are provided.

In the illustrated example, the communication terminals 266 are provided between the charging terminals 264. In other words, the charging terminal 264 is disposed outside the communication terminal 266 (in the Y direction shown in FIG. 5B). In addition, a positioning hole 268 for a connector (not shown) to be connected to the charging terminal 264 and the communication terminal 266 is provided outside each charging terminal 264 (in the opposite Y direction). In addition, the charging terminal 264 and the communication terminal 266 protrude from the side surface of the case 200, while the positioning hole 268 is a blind hole.

As shown in FIG. 5B, a distance d1 between each charging terminal 264 and the closest communication terminal 266 is wider than a distance d2 between the two communication terminals 266. In this way, it is possible to avoid short circuits between the charging terminals 264 and between the charging terminal 264 and the communication terminal 266.

As for the charging terminal 264 and the communication terminal 266, it is possible to use a material having electric conductivity, such as stainless steel, which is corrosion resistant. Further, since the charging terminal 264 and the communication terminal 266 protrude from the case 200 (such as with a convex lens-shaped convex curved surface), thus minimizing corrosion and maintaining electric conductivity. Further, the charging terminal 264 and the communication terminal 266 are provided in the side opposite the operation section 250, and therefore, the fingers operating the operation buttons 252a and 252b do not usually touch the terminals, thereby preventing contamination due to sebum or the like. In addition, the surfaces of the charging terminal 264 and the communication terminal 266 may be plated with a conductive material having corrosion resistance, for example, gold or the like, to further improve the corrosion resistance.

Returning to FIG. 3, the sensor section 280 will be described.

As shown in FIG. 3, the sensor section 280 provided with a sensor unit 282 is provided in the module 20 accommodated in the case 200. Any suitable sensor unit capable of measuring biological information, such as a pulse rate, may be used. One exemplary sensor will be described. The sensor unit 282 is a light sensor and is provided with a sensor case, and a sensor substrate with a light emitting element and a light receiving element mounted thereon. The sensor unit 282 irradiates light toward the wrist of the user from the light emitting element such as a light emitting diode (LED) and receives the light reflected by a blood vessel of the wrist by the light receiving element such as a photodiode. Such a sensor is described in detail in the present assignee's co-pending application Ser. No. 14/463,519, Attorney Docket Number 91946-082100-910013, client reference number J0173922US01, titled “Physiological Information Measuring Apparatus,” filed Aug. 19, 2014, which is hereby incorporated by reference.

A sensor bank portion 212 in which the sensor section 280 is accommodated is provided at the lid section 210. At least a portion of the sensor section 280 is provided on the sensor bank portion 212 having a disk shape recessed into the lid section 210 in the direction toward the user's skin. The sensor bank portion 212 is provided with a sensor convex portion 214 (refer to FIG. 2B).

The sensor convex portion 214 is provided so as to be pressed against the wrist or the like of the user with the measurement device 1 mounted thereon. The sensor convex portion 214 is provided with a base portion 214a extending from the lid section 210, and a tip portion 214b which is pressed against the user's skin.

It is preferable that the base portion 214a of the sensor convex portion 214 is made of an opaque material and the tip portion 214b is made of a transparent or translucent material. Further, it is preferable that the tip portion 214b has a shape which avoids irregular reflection of the light and in which the user does not feel pain at the time of putting on the device.

Therefore, in some embodiments, the base portion 214a is made of the same synthetic resin as the lid section 210 but has light-shielding coloring applied thereto. Further, the tip portion 214b is preferable arc shaped and made of transparent glass or transparent acrylic resin.

### Configuration of Band 3

The configuration of the band 3 will be described using FIGS. 1 to 3, 6A, and 6B.

The band 3 is provided in order to mount the device main body 2 on the user. The band 3 has two portions: one at each end of the device main body 2, as shown in FIGS. 1, 2A, and 2B. The first band portion 30 is mounted on a lug 203 (at the twelve o′clock side) of the device main body 2 by a mounting member 32, as shown in FIG. 3. Further, the second band portion 40 is mounted on a lug 204 (at the six o′clock side) of the device main body 2 by a mounting member 42.

A connection portion 310 which removably connects the first band portion 30 and the second band portion 40 is provided at an end of the first band portion 30 opposite the device main body 2. Further, a hook 410 which secures the second band portion 40 to the first band portion 30 is provided at an end of the second band portion 40 opposite the device main body 2.

In the following description, in the first band portion 30, the device main body 2 is described as being attached to the first end, and the side on which the connection portion 310 is provided is the second end. Similarly, in the second band portion 40, the device main body 2 side is the first end and the hook 410 is attached to the second end.

### Configuration of First Band Portion and Second Band Portion

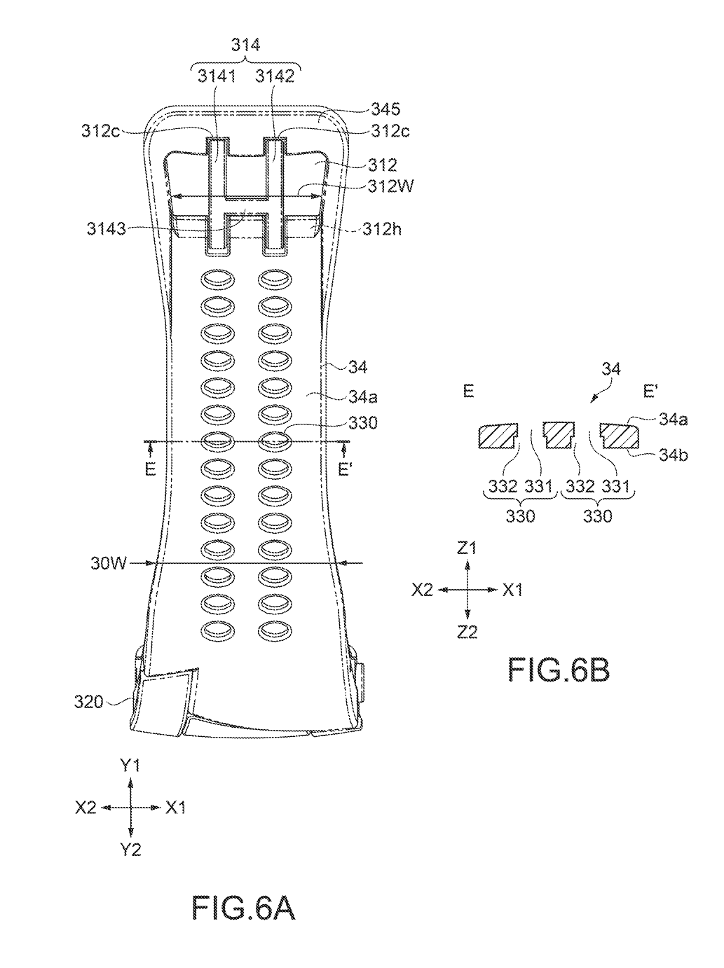

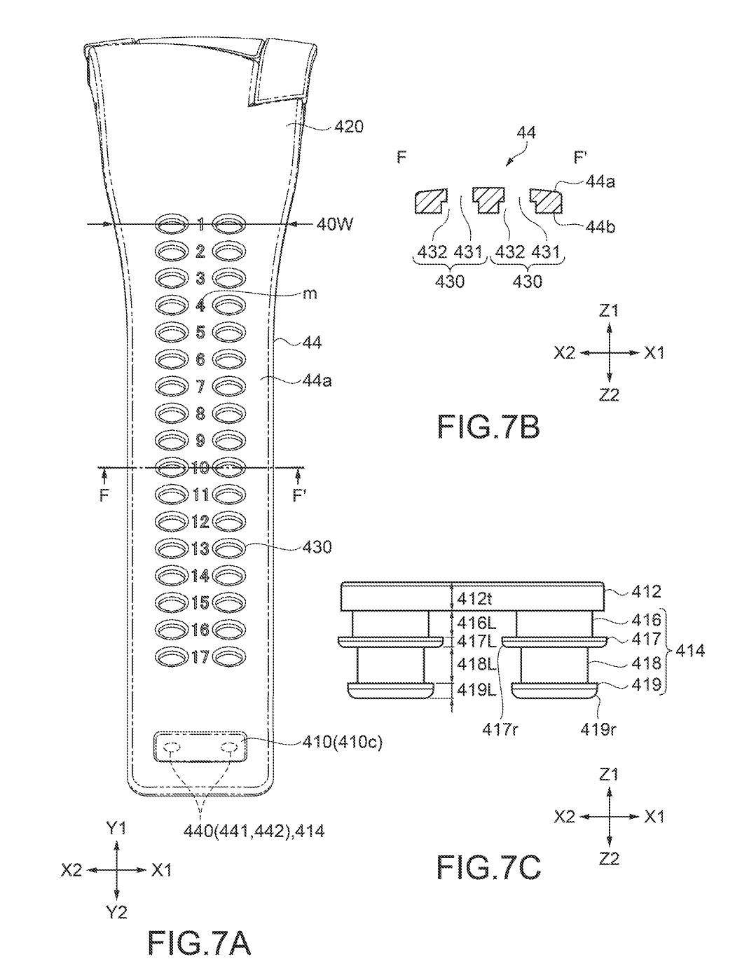

The first band portion 30 shown in FIGS. 3, 6A, and 6B has a belt portion 34, a cover portion 320 as an additional connection portion provided on the first end of the belt portion 34, and the previously mentioned connection portion 310 on the second end. The second band portion 40 shown in FIGS. 3, and 7A to 7C has the belt portion 44, the cover portion 420 as a further connection portion provided at the first end of the belt portion 44, and the hook 410 on the second end. FIG. 7A also shows a concave portion 410c in which the hook 410 is provided and a hole portion 440.

In the following description, the surface contacting the user's wrist is referred to as a back surface 34b of the belt portion 34 and a back surface 44b of the belt portion 44. The opposite, visible surface is referred to as a front surface 34a of the belt portion 34 and a front surface 44a of the belt portion 44.

### Mounting on Device Main Body 2

The first band portion 30 and second band portion 40 are mounted on the device main body 2 such that the cover portions 320, 420 cover the lugs 203, 204, respectively, with the mounting member 32, 42 sandwiched between the lug 203, 204 and the cover portion 320, 420, respectively.

The cover portions 320, 420 and the lugs 203, 204 are pivotally supported by inserting respective spring rods (not shown) into respective holes 32h, 42h provided in the mounting members 32, 42 and rod holes 203h, 204h provided in the lugs 203, 204 and locking both ends of each spring rod to locking holes 320h, 420h provided in the cover portions 320, 420, respectively.

In the band portions 30, 40, if the belt portions 34, 44, which in some embodiments are highly elastic (described later) and the device main body 2 were directly connected, there may be a concern that the connection strength thereof may not be sufficient. Therefore, the band portions 30, 40 are mounted on the device main body 2 through the cover portions 320, 420, respectively, whereby the connection can be made relatively more solidly.

In the illustrated embodiment, the connection between the device main body 2 and the first band portion 30 is performed with a miter joint such that the edge 202e of the display section cover body 202 faces the end portions 320e and 32e, which are ends of the cover portion 320 and the mounting member 32, respectively, such that the mounting member 32 and the cover portion 320 contact the edge 200e of the display window 200a. The connection between the device main body 2 and the second band portion 40 is performed with a miter joint such that the edge 202f of the display section cover body 202 faces the end portions 420f and 42f, which are ends of the cover portion 420 and the mounting member 42, respectively, such that the mounting member 42 and the cover portion 420 contact the edge 202f of the display window 200a.

Further, in the measurement device 1, a bottomed groove (i.e. a blind groove) 290 is provided between the first band portion 30 and the device main body 2 to prevent wear due to the contact of the case 200 with the mounting member 32 and the cover portion 320 when the belt portion 34 expands or contracts. An additional bottomed groove 290 is provided between the second band portion 40 and the device main body 2 to prevent wear due to the contact of the case 200 with the mounting member 42 and the cover portion 420 when the belt portion 44 expands or contracts. Further, it is possible to prevent detachment of the band portions 30, 40 due to the contact of the case 200 with the mounting members 32, 42 and the cover portions 320, 420.

### Belt Portions 34, 44

The belt portions 34, 44 are highly elastic in order to tightly mount the device main body 2 against the wrist or the like of the user. The belt portions 34, 44 may be made using a material which includes polyurethane resin or silicone resin, thereby having the stretchability and flexibility of these materials.

The belt portions 34, 44 each has a thickness in the Z direction of a central portion in a direction of a width 30W, 40W increased in a cross-section in the X direction, along line E-E′ in FIG. 6A and line F-F′ in FIG. 7A, whereby strength during expansion and contraction and during flexion are secured.

A hole 330 (in which the hook 410 of the second band portion 40 is secured) is provided in the belt portion 34. A hole 430 (in which the projecting bar 314 of the first band portion 30 is secured) is provided in the belt portion 44. The holes 330, 430 are provided in parallel in rows in the Y direction. The spacing of the holes 330, 430 in the Y direction may be constant or may become shorter toward the device main body 2, so that even users with thin wrists can finely adjust tightening.

In each hole 330, 430, as shown in FIGS. 6B and 7B, a hole portion 331, 431 on the front side and a hole portion 332, 432 on the back side are coaxial.

The holes 330, 430 each has an elliptical shape having a major diameter in the X direction and a minor diameter in the Y direction. Both the major diameter and the minor diameter are longer in the hole portions 332, 432 than in the respective hole portions 331, 431.

In addition, the shape of the holes 431 and 432 depends on the cross-sectional shape of the projecting bar 314. In the illustrated embodiment, the holes 431 and 432 are elliptical, thereby being able to be easily deformed, and thus the insertion of the projecting bar 314 can be easily performed. In addition, the holes 431 and 432 also have an excellent restoring force after the deformation.

As shown in FIG. 7A, numerical symbols m are provided between the rows of hole portions 430. The values of the symbols m increase in the Y2 direction. The symbols m are, e.g. numbers 1-17, as illustrated, or a-q. The latter may sometimes be preferred because the tenth plus symbols do not take up any more room than the first through ninth, unlike with numbers.

The symbols m allow the user to identify his or her unique optimum position for inserting the projecting bar 314 at a glance. Further, the symbol m is positioned between rows of hole portions 430, and therefore, additional space is not necessary.

As shown in FIG. 7A, the belot portion 44 further includes a concave portion 400c in which a base portion 412 of the hook 410 is fitted. The belot portion 44 also includes the hole portion 440 in which a pin 414 extending from the base portion 412 is inserted.

The concave portion 400c has a concave shape conforming to an outer peripheral edge of the base portion 412 on the front surface 44a of the belt portion. Further, a depth 400d of the concave portion 400c is approximately the same dimension as a thickness 412t of the base portion 412.

The hole portions 440 are provided in parallel in the X direction and coaxially with the pins 414 extending from the base portion 412, as shown in FIG. 7A. In the hole portion 440, a hole (not shown) provided in a bottom portion of the concave portion 400c on the front surface 44a and a hole (not shown) in the back surface 44b are coaxial.

### Connection Portion 310

The connection portion 310 is provided on the second end of the belt portion 34.

A thick portion 345 whose thickness is higher than at a portion in which the holes 330 are provided is provided at the second end of the belt portion 34. The connection portion 310 is provided at the thick portion 345. An insertion hole 312 into which the second band portion 40 is inserted and a projecting bar 314 which is inserted into a hole portion 430 (refer to FIGS. 7A to 7C) provided in the second band portion 40 are provided at the connection portion 310.

The band insertion hole 312 has a width 312W in the X direction which is wider than a width 40W (refer to FIG. 7A) of the second band portion 40.

Further, the width 312W is tapered, and is wider at the Y1 side than the Y2 side.

It is therefore possible to easily insert the second band portion 40 in the Y1 side of the insertion hole 312. Further, it is possible to tighten the band by pulling the second band portion 40 to the Y2 side.

The projecting bar 314 is pivotally supported on the belt portion 34 by a spring rod (not shown) which is inserted into the locking hole 312h at the Y2 side of the band insertion hole 312, and a hole (not shown) in the projecting bar 314. A portion of the projecting bar 314 is fitted into the concave portion 312c at the Y1 side of the band insertion hole 312, whereby the projecting bar 314 is locked to the belt portion 34.

Two bar portions 3141 and 3142 extend in the Y1 direction from the projecting bar 314. A connection bar 3143 extends in the X direction. The cross-sectional shape of each of the bar portions 3141 and 3142 is flat. The projecting bar 314 has a flat shape, whereby it is possible to increase a contact area when having been inserted into the hole portion 430, prevent the hole portion 430 from being extended, and prevent a shift of the measurement device 1 when worn by the user. Thus, the projecting bar 314 has an H-shape, and thus it is possible to easily insert the projecting bar 314 into the hole portions 430, while maintaining an interval between the bar portions 3141 and 3142 by the connection bar 3143.

The material of the projecting bar 314 is not particularly limited, but it should have toughness capable of withstanding the restoring forces of the belt portions 34 and 44, and corrosion resistance. Stainless steel is one example. Further, the projecting bar 314 is subjected to hairline machining along the Y direction to increase is visibility, thereby aiding insertion into the hole portion 430.

### Hook 410

As shown in FIGS. 1 to 3 and FIGS. 7A to 7C, the hook 410 is provided on the second end of the belt portion 44.

As shown in FIG. 7C, the hook 410 is provided with the base portion 412 and the pins 414 extending from the base portion 412. The pin 414 includes a first shaft 416 and a second shaft 418. The hook 410 locks the second band portion 40 to the first band portion 30 by inserting the pins 414 into the holes 330 of the first band portion 30.

The pins 414 are spaced apart at the same interval as the holes 330 of the belt portion 34 in the X direction, and the cross-sectional shape is elliptical to match the shape of the hole 440, i.e. has a major diameter in the X direction and a a minor diameter in the Y direction.

The first shaft portion 416 protrudes from the base portion 412 at the X1 end and the second shaft portion 418 protrudes from the base portion 412 at the X2 end.

A first shoulder 417 is provided on the first shaft portion 416 in the Z2 direction from the base portion 412. The first shoulder 417 has a curved surface 417r at the Z2 edge.

The cross-sectional area of the first shoulder 417 is larger than that of the first shaft portion 416, larger than the hole 441, and smaller than the hole 442.

Further, a second shoulder 419 is provided on the second shaft portion 418 in the Z2 direction from the first shaft portion 416. The second shoulder 419 has a curved surface 419r at the Z2 edge.

The cross-sectional area of the second shoulder 419 is larger than that of the second shaft portion 418, larger than the hole 331, and smaller than the hole 332.

The thickness in the Z direction of the base portion 412 and the length in the Z direction of the pin 414 are determined depending on the depths of the hole portion 440 and the hole 331 (the hole 330).

The thickness 412t of the base portion 412 is approximately the same as the depth of the concave portion 400c. A length 416L of the first shaft portion 416 is approximately the same as the depth of the hole 441. The thickness 417L of the first shoulder 417 is approximately the same as the depth of the hole 442. The length 418L of the second shaft portion 418 is approximately the same as the depth of the hole 331. The length 419L of the second shoulder 419 is approximately the same as the depth of the hole 332.

The base portion 412 is fitted into the concave portion 410c. In addition, the pins 414 are inserted into the hole portions 440. The second shaft portions 418 protrude from the back surface 44b of the belt portion 44, thereby being able to be inserted into the holes 330 in the first band portion 30.

In this way, in the first shaft portion 416 inserted into the hole 441, the first shoulder 417 is caught in the hole 441, and thus the hook 410 is secured to the belt portion 44.

More specifically, due to the curved surface 417r of the first shoulder 417, contact resistance when inserting the first shaft portion 416 into the hole 441 is low. In addition, the curved surface 417r is not provided at the first shoulder 417 on the base portion 412 side, and therefore, when extracting the first shaft portion 416 from the hole 441, a larger force than the force at the time of insertion is required. Therefore, the hook 410 is secured to the belt portion 44 until the user purposely removes it.

Further, the second shoulder 419 is caught in the hole 331 in the first band portion 30, thus securing the second band portion 40 to the first band portion 30.

More specifically, due to the curved surface 419r of the second shoulder 419, contact resistance when inserting the second shaft portion 418 into the hole 331 is low. In addition, the curved surface 419r is not provided at the second shoulder 419 on the first shaft portion 416 side, and therefore, when extracting the second shaft portion 418 from the hole 331, a larger force than the force at the time of insertion is required. Therefore, the second band portion 40 is secured to the first band portion 30 until the user purposely removes it.

### Cover Portions 320, 420

The cover portion 320 is disposed at the Y2 side of the belt portion 34, and has the locking hole 320h provided therein. The cover portion 420 is disposed on the Y1 side of the belt portion 44, and has the locking hole 420h provided therein.

The cover portion 320 connects the first band portion 30 to the device main body 2 through the mounting member 32. The cover portion 420 connects the second band portion 40 to the device main body 2 through the mounting member 42. A width 320W of the cover portion 320, a width 420W of the cover portion 420, and a width 2W of the device main body 2 are all approximately equal (in the X direction).

Further, the cover portion 320 has the oblique end portion 320e which parallels the edge 200e of the non-rectangular parallelogram shaped display window 200a (refer to FIG. 3). The cover portion 420 has the oblique end portion 420f which parallels the edge 200f of the display window 200a. In this way, the device main body 2, the first band portion 30, and the second band portion 40 appear integrally formed, and thus it is possible to alleviate a feeling of pressure due to mounting. Further, since the device main body 2 does not protrude from the band portions 30, 40 in the width direction (the X direction), the clothes of the user are prevented from being caught on the device main body 2 at the time of putting on the apparatus.

According to the embodiments described above, the following effects can be obtained.

According to the measurement device 1, the device main body 2 and the band 3 are connected by a miter joint, so their widths appear to blend seamlessly. Therefore, it is possible to make the device main body 2 appear large, since it blends directly into the band, and thus the user perceives a low pressure during prolonged wearing. Therefore, the user wears the device 1 tighter for a longer time than he or she otherwise might, and thus it is possible to obtain biological information with high precision. Further, the shape of the measurement device 1 changes continuously and smoothly, whereby, even when the user is in motion with the device main body 2 mounted on his or her wrist, it is possible to prevent the device main body 2 from being caught in a sleeve. Also, by continuing to measure the biological information over a long period of time, the user can more accurately glean the relevant information.

In the detailed description above, an example having a function of measuring physiological or other biological information has been described. However, the invention is not limited thereto, and the device 1 can collect and display a variety of information. By modifying the module 20, the device 1 may be, for example, a navigation device, a communication device, or even a simple wristwatch.

What is claimed is:

1. A biological information measurement device, comprising:
a device main body configured to contact a skin surface of a user to thereby acquire biological information about the user, wherein the device main body presents a non-rectangular parallelogram surface in plan view, the parallelogram surface comprising two longitudinal edges disposed along a longitudinal direction, and two oblique edges disposed along an oblique direction that is transverse to, but not perpendicular to, the longitudinal direction; and
a band attached to the oblique edges and generally extending in the longitudinal direction, wherein the band is configured to secure the device to a body part of the user.

2. The biological information measurement device according to claim 1, further comprising a mounting member which connects the device main body and the band.

3. The biological information measurement device according to claim 1, further comprising a bottomed groove disposed between the band and one of the oblique edges of the device main body.

4. The biological information measurement device according to claim 1, wherein the device main body comprises:
a case comprising a front surface configured to be viewed by the user and a rear surface configured to contact the user's skin;
a module comprising an electronic circuit, wherein the module is disposed in the case; and
a film comprising metal, disposed on the front surface of the case, wherein the film and the module are electrically connected.

5. The biological information measurement device according to claim 4, wherein the module comprises a first display portion presenting a rectangular surface in plan view, the rectangular surface comprising two longitudinal edges which are collinear with respective ones of the longitudinal edges of the parallelogram surface, and two lateral edges disposed perpendicular to the longitudinal direction.

6. The biological information measurement device according to claim 5, wherein each of the lateral edges of the rectangular surface defines an acute angle at an intersection with a respective one of the oblique edges of the parallelogram surface.

7. The biological information measurement device according to claim 5, wherein the module further comprises a second display portion adjacent the first display portion.

8. The biological information measurement device according to claim 7, wherein the second display portion presents a right triangular surface in plan view.

9. The biological information measurement device according to claim 4, wherein the module comprises:
a control section; and
an operation section connected to the control section, wherein the operation section is manipulable by the user and is disposed on one of the longitudinal edges.

10. The biological information measurement device according to claim 4,
wherein the module comprises:
a control section; and
a power supply section comprising a battery which drives at least one of the control section, the first display portion, and the second display portion;
wherein the device further comprises:
at least one output terminal configured to output data from the control section to outside the device, connected to the module and disposed on one of the longitudinal edges; and
at least one charging terminal configured to charge the battery, connected to the module and disposed on one of the longitudinal edges.

11. The biological information measurement device according to claim 10,
wherein the at least one output terminal comprises a first output terminal and a second output terminal;
wherein the at least one charging terminal comprises a first charging terminal and a second charging terminal;
and wherein a first distance between the first and the second charging terminals is different than a second distance between the first and the second output terminals.

12. The biological information measurement device according to claim 11, wherein the terminals are disposed along the longitudinal edge in a row in the following order: the first charging terminal, the first output terminal, the second output terminal, the second charging terminal.

13. The biological information measurement device according to claim 10, wherein each of the terminals comprises stainless steel.

14. The biological information measurement device according to claim 10, wherein tips of the terminals are exposed on a surface of the case.

15. The biological information measurement device according to claim 10, wherein the terminals are disposed along the longitudinal edge in a row, and wherein the case further comprises with a positioning hole disposed in the same row.

16. A method of measuring biological information of a user, comprising:
measuring the biological information using a device that closely contacts the user's skin; and
presenting a non-rectangular parallelogram surface of the device to the user to thereby alleviate a sense of discomfort caused by the perception of excessive pressure.

17. The method of claim 16, wherein the device comprises a case comprising a front surface configured to be viewed by the user and a rear surface configured to contact the user's skin, and a module comprising an electronic circuit, wherein the module is disposed in the case, the method further comprising:
presenting a rectangular surface of a first display portion of the module to the user, the rectangular surface comprising two longitudinal edges which are collinear with respective longitudinal edges of the parallelogram surface, and two lateral edges disposed perpendicular to a longitudinal direction.

18. The method of claim 17, wherein each of the lateral edges of the rectangular surface defines an acute angle at an intersection with a respective oblique edge of the parallelogram surface.

19. The method of claim 17, further comprising presenting a second display portion of the module to the user, wherein the second display portion is adjacent the first display portion.

20. The method of claim 19, wherein presenting the second display portion comprises presenting a right triangular surface to the user.
